(19) United States
(12) Patent Application Publication
(10) Pub. No.: US 2007/0179283 A1
(43) Pub. Date: Aug. 2, 2007

(54) Extraction process for a pharmaceutical product

(76) Inventor: Bhanu Manickavasagam, Chapel Hill (AU)

Correspondence Address: MOORE & VAN ALLEN PLLC, P.O. BOX 13706, Research Triangle Park, NC, 27709, US

(21) Appl. No.: 10/554,865

(22) PCT Filed: Apr. 30, 2004
(86) PCT No.: PCT/AU04/00574

§ 371 Date: Dec. 19, 2006

Related U.S. Application Data
(60) Provisional application No. 60/478,654, filed on Jun. 13, 2003.
(60) Provisional application No. 60/542,438, filed on Feb. 6, 2004.

(30) Foreign Application Priority Data
May 1, 2003 (AU) 2003902066

(51) Int. Cl. C07K 14/78

(52) U.S. Cl. 530/356

(57) ABSTRACT

A process for isolating a soluble, native collagen from a marine invertebrate, comprising the steps of: 1) treating a collagen-containing portion of the marine invertebrate with a weak acid solution in order to solubilise native collagen fibrils; 2) centrifuging the resultant slurry to remove tissue particulates; 3) adjusting the pH of the supernatant in order to precipitate collagen by addition of a base; 4) collecting the precipitated collagen; 5) resuspending the precipitated collagen; and performing buffer exchange against water using an 15 ultrafiltration membrane.

### TECHNICAL FIELD

The present invention is concerned with a process for obtaining native collagen through extraction from marine invertebrates. A soluble native collagen is obtained, which is particularly suited to pharmaceutical use as an alternative product to land animal collagen due to the current concerns about Bovine Spongiform Encephalopathy (BSE) or Mad Cow Disease, but may also be put to other uses in place of land animal collagen as well as converted into gelatin by heating.

### BACKGROUND ART

BSE is an extremely serious disease of cattle, considered to originate from infected meat and bone meal in cattle feed concentrates. BSE is transmissible in cattle, and was first identified in United Kingdom in 1986. It is invariably fatal. There is no treatment and it is difficult to detect. Recent research indicates that humans who eat infected meat could develop Creutzfeldt-Jacob Disease (CJD), the human equivalent of the cattle disease. At least 10 CJD patients in Britain are believed to have contracted the disease from eating beef. Most people who develop CJD are aged between 50 and 70.

Currently the culling of the cattle is of primary importance in the United Kingdom and Europe to safeguard the herd. Nevertheless, BSE poses a significant threat to the future supply of bovine meat and dairy products for the human and animal food consumption, and to the supply of important bovine by-products used in the pharmaceutical, medical and cosmetic industries. Presently, the manufacturers of pharmaceuticals across Japan, UK and Europe and other countries have stopped using British beef and beef products in the manufacture of pharmaceuticals and medicines as well as cosmetics products to prevent the spread of “Mad Cow” disease to humans. Also imports of medicine and cosmetics containing substances from British cows have stopped.

The most widely used bovine product is collagen. Collagen is a fibrous protein which comprises most of the white fibre in the connective tissues of mammals, particularly the skin, tendon, bone and muscles. A number of different vertebrate collagen have been identified, up to 19 groups so far have been identified in vertebrates (Prockop and Kivirikko, 1995) of which type I, II and III represent the most widely distributed types. Collagen comprises about 30% of the total organic matter in mammals and nearly 60% of the protein content. Collagen is deposited rapidly during periods of rapid growth, and its rate of synthesis declines with age, particularly in tissues that undergo little remodeling.

The collagen molecule is built from three peptide chains which are helical in conformation. The helix extends through 1014 residues per chain (Hoffmann et al 1980). At the end of the triple helical domain, short non-helical chains, namely telopeptides, having a non-repeating sequence and spanning from 9 to 25 residues, extend beyond the triple helix from both ends of each chain (Hoffman et al, 1980). The telopeptide portions of native collagen are believed to be the major sites of its immunogenicity and have been shown to play a crucial role in directing fibrillogenesis (Helseth and Veis 1981). The length of the helix and the nature and size of nonhelical portions of the molecule vary from type to type. If the triple helical structure of the collagen molecule is destroyed by heat, the properties of the polypeptides change entirely in spite of having the same chemical composition.

In skin, collagen exists as fibres which are woven into networks constituting fibre bundles, the fibres being maintained in the bundle by interfibrillar cement. Collagen fibrils typically have a length of about 2 mm while the fibres are naturally much longer and of greater diameter.

Vertebrate collagen has a molecular weight of 300,000 Daltons. Each strand of the triple helix has a molecular weight of approximately 100,000 Daltons and assumes a left-handed helix configuration (Lehninger 1975). Most vertebrate collagens present in skin, tendon, muscle, and bone are composed of two identical and one different a chains denoted by [(α1)₂α2] (Piez et al. 1963; Lewis and Piez, 1964; Miller et al, 1967; McClain et al. 1970) except for codfish skin and chick bone collagen which contains three different chains [(α1) (α2) (α3)] (Piez, 1965; Francois and Glincher 1967). Cartilage collagen has in addition to molecules of chain composition [(α1)₂α2], another type of molecule which is composed of three identical chains, [α1 (II)₃] (Miller 1971; Trelstad 1970). The α1 (II) chain is apparently different from the α1 chain, which is designated α1 (I) only when compared to α1 (II), in its high content of glycosylated hydroxylysines. The collagen present in basement membranes (Kefalides, 1971) and sea anemone body wall (Katzman and Kang 1972) have also been confirmed to consist of identical a chains.

Collagen is the only mammalian protein containing large amounts of hydroxyproline and it is extraordinarily rich in glycine (approximately 30%) and proline. The hydroxyproline is essential for the formation of hydrogen-bonded water-bridges through the hydroxyl group and the peptide chain, thereby stabilising the triple helix. In soluble collagen the inter-molecular bonds have been cleaved, but leaving the triple helices intact.

Collagen type I, especially bovine skin collagen, has been utilised in foods and beverages, cosmetics and medical materials. Purified adult bovine collagen is used in a variety of medical devices, including hemostats, corneal shields, and for soft tissue augmentation. Collagen gels are often intermediates in the preparation of these devices and, in some cases, the gels represent the final medical products. There are also collagen masks or face-packs intended for use on the skin, both for therapeutic and cosmetic purposes. Purified calf skin collagen is an important biomaterial used in several devices as prostheses, artificial tissues, material for construction of artificial organs and as a drug carrier because the collagen molecule is non-toxic toward an organism and has a high mechanical strength. It is also useful in cosmetic compositions for the same reason.

In the biomedical field natural fibres are used in sutures and ligatures. A ligature is a thread used to tie off a bleeding vessel, while a suture is used to sew up a wound. The wound may be internal or it may be exposed. The sutures used for closing an internal wound are less easily removed. Thus an absorbable (or biodegradable) material offers a distinct advantage.

As the collagen becomes increasingly cross-linked it also becomes less hygroscopic. One of the effects of ageing in mammals is an increase in the cross-linking of collagen molecules. As cross-linking increases, it becomes more and more difficult to extract tropocollagen from mammalian sources. Uncross-linked tropocollagen has been used in cosmetics because of its association with unwrinkled skin.

Vertebrate collagen generally has to be purified extensively to remove all non-collagenous, contaminating structures. The final product of most collagen isolation and purification procedures, which consist mainly of enzymatic degradation of the non-collageneous component of connective tissue, are monomeric collagen molecules. When these rods are reconstituted into films, membranes, or sponges they will contribute very little to the mechanical strength of the final structure. It would be desirable in a purification procedure to preserve the natural structure of collagen fibres and fibrils. Due to the length (2-10 cm) and thickness (40μm) of these highly pure collagen fibres, they can be further processed into threads, sutures or non-woven fleece layers, and may be knitted or woven.

Two methods have been applied to solubilise the highly cross-linked collagen tissue in vertebrates in conventional practice. These are (1) digestion with proteolytic enzymes and (2) treatment with alkali.

Proteolytic digestion with enzymes such as pepsin is often used because of the relative ease with which the cross-links in collagen may be broken. Pepsin is the most commonly used enzyme because it is available in pure form from commercial sources and can be employed in an acidic solvent in which the monomer molecules readily dissolve. Although limited proteolysis with pepsin has been extremely useful in preparing relatively large amounts of the various collagens in essentially monomeric form from a number of animal and human tissues, the procedure has its limitations. For example, the molecules are obtained with altered nonhelical extremities, and this effectively precludes subsequent studies designed to evaluate the structure and function of these regions. Furthermore, since enzyme-solubilised collagen is rich in monomeric collagen but without telopeptides, collagen fibril reconstruction is greatly inhibited and reconstructed fibrils show low thermal stability as compared with soluble collagen with telopeptides.

Collagen hydrolysates prepared from native collagen by enzymatic hydrolysis to form peptides exhibit molecular weights in the range of 1,000 to 10,000 Daltons. In vertebrate tissue the process takes at least 2-3 days for complete extraction at 4° C.

Alkaline treatment is usually performed by immersing collagenous tissues in a 2-5% sodium hydroxide solution containing sodium sulphate and amines as a stabiliser and a nucleophile, respectively, at 4-20° C. for several days. The tissue is then further treated with acid. It is a time-consuming process which takes up to several months, depending on environmental temperatures. Traditionally bovine hide has been conditioned by an alkaline liming process, which takes many weeks. The alkaline treatment modifies the protein by partly removing amine and amide groups. Most of the swelling and hydrolysis of amide groups occurs during the early stages of liming, and there is noticeable evolution of ammonia as the collagen isoelectric point falls near pH 5.

International Publication No. WO02/102831 describes a process for isolating a collagen-derived protein fraction from a marine invertebrate through treating a collagen containing portion thereof with a weak acid solution in order to solubilise a collagen-derived protein fraction. A native collagen is precipitated from the weak acid solution by salting out with 0.3M sodium chloride. The precipitate must then be treated to remove the excess sodium chloride by dialysis against de-ionised water. It is then dialysed against a weak acid solution to adjust the pH and a solid product is isolated by freeze drying. It has been found that the product obtained as a result of this process varies greatly in quality.

In general, there is no satisfactory way to purify native insoluble collagen fibrils, especially from a tissue in which the collagen is highly cross-linked in order to produce a soluble, native collagen.

### DISCLOSURE OF THE INVENTION

The present invention is based on the unexpected finding that a modification of the process described in WO02/102831, the contents of which are incorporated herein by reference, results in the isolation of a soluble, native collagen. It was surprisingly found that when collagen was precipitated by way of pH change instead of salting out, and that step was followed with buffer exchange, a soluble native collagen was obtained.

Accordingly, in a first aspect the present invention provides a process for isolating a soluble, native collagen from a marine invertebrate, comprising the steps of:

1) treating a collagen-containing portion of the marine invertebrate with a weak acid solution in order to solubilise native collagen fibrils;

2) centrifuging the resultant slurry to remove tissue particulates;

3) adjusting the pH of the supernatant in order to precipitate collagen by addition of a base;

4) collecting the precipitated collagen;

5) resuspending the precipitated collagen; and

6) performing buffer exchange against water using an ultrafiltration membrane.

It is observed that the pH precipitation process results in a precipitate of significantly different texture to that isolated by salting out. While not wishing to be bound by theory, it is believed that the more homogenous nature of the precipitate may allow more complete buffer exchange and so allow better removal of salts which may contribute to insolubility.

It is preferable that the weak acid solution is an acetic acid solution, typically a 3% solution. A weak acid is one with a dissociation constant between 1.0×10⁻⁵ and 1.0×10⁻² in aqueous solution and so is predominantly un-ionised, and these may be readily identified by the person skilled in the art but include lactic, butyric, formic, propionic and citric acids.

Advantageously, the pH adjustment takes place after the collagen-containing portion has been in contact with the weak acid solution for 1 to 20 days in a coldroom, preferably 3 to 6 days, most preferably 6 days.

Typically the weak acid solution is subjected to some form of agitation during the extraction process described above. Preferably, the collagen-containing portion is suspended in the weak acid solution, and the suspension is stirred in order to ensure good yield and high product quality.

Typically the marine invertebrate is prepared for extraction by mechanical disruption of the collagen-containing portion.

Advantageously, the collagen-containing portion is muscle tissue, which has preferably had pigment removed therefrom. This may be achieved by soaking the intact muscle tissue in a weak acid solution. The weak acid solution is typically an acetic acid solution, preferably a 0.2M solution.

In a particularly preferred embodiment of the invention, the marine invertebrate is abalone. Preferably the abalone is a commercial species such as the black-lip abalone, Haliotis ruber, the brown-lip abalone Haliotis conicopora and the green-lip abalone, Haliotis laevigata, or Roe's abalone, Haliotis roei.

Advantageously, the collagen-containing portion is spun down in a centrifuge and native collagen is precipitated from the supernatant. Additional collagen may be extracted from the pellet, if desired. In a preferred collection process from the supernatant, sufficient base, typically 1M NaOH, is added to bring the supernatant to a pH of 4.5 in order to precipitate the collagen fibrils.

Advantageously, collagen precipitation takes place over a period of 1 to 10 hours in a coldroom, preferably 2 to 6 hours, most preferably 3 hours. Typically the mixture is stirred continuously during precipitation.

The precipitated collagen is typically collected by centrifugation.

The precipitated collagen may be resuspended in de-ionised water, typically with pH adjustment to 3.5 with any suitable acid. The resuspended collagen precipitate is buffer exchanged against water, typically de-ionised water, using an ultrafiltration membrane, typically a 100 kD NMCO ultrafiltration membrane.

Advantageously, the buffer exchanged product is freeze dried to collect the soluble, native collagen in solid form.

Gelatin is a protein derived from collagen. When collagen is heated at a certain temperature the collagen molecule undergoes a helix coil transition. The helix unfolds and the collagen becomes more readily soluble. The temperature at which this occurs depends upon the amount of proline and hydroxyproline in the α chain, and this temperature is the point of denaturing. For deep cold water fish collagen, this temperature is approximately 15° C. while for bovine collagen it is approximately 40° C. At a certain temperature the collagen in the raw skin will relax and the skin will shrink (shrinkage temperature) . The amount of imino acids, proline and hydroxyproline, determines the shrinkage temperature and the denaturing temperature. It has been found that by heating collagen of the present invention gelatin can be produced.

The polypeptides of the present invention are proposed for use in place of collagen isolated from land vertebrates, either through use of the collagen itself or following a subsequent treatment such as conversion to gelatin. Collagen is useful in a wide variety of fields, in native form or once treated. For example, collagen may be used as a cosmetic ingredient, in the form of injectable collagen, in biomedical devices, as a pharmaceutical substance and in food products and beverages. In particular, collagen finds use as a surface treatment in cell culture. Collagen prepared by the present invention may be used as a direct substitute for land animal collagen, and its manner of use in each of these fields would be well understood by the person skilled in the art. Gelatin is useful at least in the form of edible gelatin and as a flocculating agent in beverages, in industrial uses such as the manufacture of PVC pipes, glue and carbonless paper, as photographic gelatin for emulsion formulation, as a capsule coating for pharmaceuticals and as an ingredient in cosmetics, in like manner to gelatin obtained from land animals. It may be expected that collagen or the products derived from treatment thereof have equivalent or superior performance to the corresponding land animal collagen. For example, cells grown on abalone culture have shown a measurably higher growth rate than those grown on bovine collagen, and appear to exhibit greater organisation. Accordingly, it is particularly preferred to use abalone collagen as a surface treatment in cell culture.

### BRIEF DESCRIPTION OF THE DRAWINGS

FIG. 1 is a graph of cell count versus time as growth occurs of cell cultures on collagen coated surfaces and an uncoated plastic control surface;

FIG. 2 is an image produced by phase contrast microscopy of cell culture growth

top to bottom—days 1 to 4

left to right—C control, BV bovine collagen, AB abalone collagen; and

FIG. 3 is an image from phase contrast microscopy of long term cell culture growth (day 23)

left to right—C control, AB abalone collagen.

### MODES FOR CARRYING OUT THE INVENTION

### Example 1

### Isolation and Purification

1^{st} Extraction

Step 1.

Live abalone were obtained and transferred to a holding tank controlled at 10° C.

Step 2.

Abalone were removed from the tank as required.

Step 3.

The abalone were rinsed under running water prior to shucking. Working on a chopping board, the animals were shucked with a spatula to remove the body from the shell. The shell was stored for later use.

Step 4.

The guts were removed by carefully cutting around the top of the foot with a scalpel and stored for later use.

Step 5.

The mouth area was cut away using a scalpel and stored for later use.

Step 6.

The pigmentation from the foot area and adductor area was removed by soaking overnight with gentle agitation in 0.2M acetic acid and then scrubbing with a stiff bristled brush under running water.

Step 7.

The whole muscle tissue was cut into 1-2″ pieces using a scalpel or knife.

Step 8.

The tissue was blended by passage through a Comitrol 3600. The Comitrol is used for size reduction of the raw material prior to extraction. It works by forcing material through a stationary cylindrical screen or cutting head with a three-bladed rotor. The cutting head has blades 0.03 inches thick and spaced 0.06 inches apart. Hold-up in the cutting head was flushed through with a few ice cubes. The blended tissue was weighed.

Step 9.

The blended tissue was added to a 3% acetic acid solution (pH 3.0). The volume of the acid solution was 12 ml/g of blended tissue.

Step 10.

The slurry was stirred in a coldroom for six days to extract native collagen fibrils.

Step 11.

The slurry was centrifuged at 3,500 g, 4° C. for 20 minutes to remove tissue particulates. The pelleted tissue was retained.

Step 12.

The pH of the supernatant was gradually adjusted to 4.5 by addition of 1 M NaOH in order to precipitate the collagen. The mixture was kept in a coldroom with constant stirring for 3 hours.

Step 13.

The precipitated collagen was collected by centrifugation at 5,000 g, 4° C. for 10 minutes.

Step 14.

The precipitated collagen was resuspended in a minimum quantity of de-ionised water and the pH lowered to 3.5 with 1 M HCl.

Step 15.

The collagen suspension was diluted ½ or 1:1 with de-ionised water and then buffer exchanged against 2 volumes of de-ionised water using a 100 kD NMCO ultrafiltration membrane.

Step 16.

The buffer exchanged collagen was poured into freeze drying trays, placed into a freeze dryer, and frozen to −20° C. by refrigeration of the freeze dryer shelves.

Step 17.

The collagen was freeze dried to a final product temperature of 20° C. This took approximately 48 hours.

Step 18.

The freeze dried collagen was milled using the Comitrol.

Step 19.

The milled collagen was stored for analysis.

2^{nd} Extraction

The pelleted tissue from Step 11 of the 1^{st} extraction was added to a 3% acetic acid solution (pH 3.0) for re-extraction. The volume of the acid solution was 5 ml/ml of pelleted tissue.

The slurry was stirred in a coldroom for three days. The re-extraction then proceeds as from Step 11 of the 1^{st} extraction.

Analysis of Freeze Dried Collagen

1. Appearance

Note was made of the colour, odour, texture of the material by visual inspection.

2. Solubility

The solubility of freeze dried material was tested at a concentration of 0.1% in 0.1M acetic acid at room temperature. The clarity of the solution was observed after stirring for 3 hours.

3. Molecular Weight, Purity and Chain Type Composition

The molecular weight, purity and chain type composition of abalone collagen was evaluated by sodium dodecyl sulphate polyacrylamide gel electrophoresis (SDS-PAGE) 12% Gradipore iGel precast Tris glycine gels were used. SDS-PAGE was performed according to the method of Laemmli (1970).

Freeze dried abalone collagen was dissolved at 1 mg/ml in 0.1M acetic acid. Samples were then diluted ½ with Gradipore Glycine sample buffer and the pH adjusted with 1M NaOH.

The samples were then placed into a boiling water bath for 3 minutes then allowed to cool. The gel was assembled in a Biorad Mini-Protean 3 electrophoresis cell. The inner chamber was filled with SDS glycine running buffer and the samples loaded with an autopipettor and standard yellow tips. The total protein load per well was 2 μg. A molecular weight marker (Biorad broad range prestained marker) was run with each gel. The outer chamber was filled with running buffer to the level of the wells.

The running conditions were 150 V constant voltage over 60 minutes with an approximate start current of 50 mA. The gel was then removed from the casing and rinsed with water for around 30 seconds. The gel was stained with around 50 ml of Gradipore Gradipure stain (based on colloidal G-250 Coomassie blue) overnight with gentle shaking. The gel was destained with frequent changes of water. Bands were generally visible after 5 minutes with about a day required for complete destaining.

Permanent storage of gels was achieved by drying between cellophane sheets. The destained gels were soaked in a drying solution of 20% methanol and 2% glycerol with gentle shaking for 15 minutes. Two cellophane sheets per gel were wetted in the drying solution for around 30 seconds. The trimmed gel was clamped between the cellophane sheets in a drying frame and left to stand in an open container at room temperature for 2 days. The gel was then pressed for a number of days to prevent curling.

A log plot is made of molecular weight versus distance migrated down the gel for the molecular weight standard and a linear trendline determined using MS Excel. The formula generated can then be used to calculate the molecular weight of the sample bands according to their migration distance.

Results

1. Appearance of Freeze Dried Native Abalone Collagen—(Table 1)

TABLE 1

| | |
|---|---|
| Appearance | off white powder, negligible odour |

2. Solubility of Freeze Dried Native Abalone Collagen—(Table 2)

TABLE 2

| | |
|---|---|
| Solubility in 0.1M Acetic Acid | soluble at 0.1% |

3. Molecular Weight, Purity and Chain Type Composition of Native Abalone Collagen—(Table 3)

TABLE 3

| Purity | Chain Composition | Molecular Weight kDa |
|---|---|---|
| single band | α1 | 111 |
| | α2 | 103 |

### Example 2

### Cell Culture

Step 1.

Freeze dried type 1 collagen was dissolved at 1-2 mg/ml in 0.1M acetic acid.

Step 2.

The collagen solution was sterilized by gently layering a 10% volume of chloroform on the bottom without mixing and allowing to stand overnight in a coldroom.

Step 3.

The top (collagen) layer was aseptically removed and transferred to a sterile vessel.

Step 4.

The growth surface of the culture vessel (24-well plate) was rinsed with 0.1 ml/cm² (200μl) of sterile filtered 0.2 g/l EDTA.4Na.

Step 5.

The wells were coated with 10 μg/cm² of collagen solution and spread out to cover the growth surface by repeated aspiration with the pipette tip.

Step 6.

A row of 6 wells was left uncoated as a control while other rows were coated with abalone collagen and calf skin collagen respectively.

Step 7.

The coated plate was incubated at 37° C. for 4-5 hours then sanitised by standing under UV light overnight.

Step 8.

Excess coating solution was aspirated and the wells rinsed with basal medium (Ham's Nutrient Mixture F12).

Step 9.

Subcultured mammalian cells (CHO Kl) were resuspended in F12+10% FCS+1% penicillin/streptomycin and counted by hemacytometer with viability by trypan blue exclusion.

Step 10.

Cells were seeded at around 5×10⁴ cells/ml and incubated at 37° C. under 5% CO₂.

Step 11.

Cultures were examined daily for morphological differences using an inverted microscope. Photographic records were made with a digital camera.

Step 12.

Growth of the cultures was measured daily by harvesting the cells and counting with a hemacytometer.

Step 13.

Harvesting of the cells from the control wells was by trypsinisation. The culture media was removed and the well rinsed with 0.15 ml/cm² (300 μl) of basal medium. An equal volume of 0.25% trypsin/EDTA solution was used to detach the cells. Incubation was for 4 minutes at room temperature. Cells were resuspended to a final volume of 1 ml using basal medium and immediately counted.

Step 14.

Harvesting of the cells from the coated wells was by incubation with collagenase (0.1% in basal medium). The culture media was removed and the well rinsed with 0.2 ml/cm2 (400 μl) of basal medium. An equal volume of collagenase was added to the well and mixed by repeated aspiration with the pipette tip. The plate was then alternately incubated at 37° C. for 10 minutes, mixed for 5 minutes, incubated at 37° C. for 10 minutes, mixed for 5 minutes, then incubated at 37° C. for 20 minutes. The resuspended cells were diluted with basal medium to a volume of 1 ml for counting.

Alternatively 0.3% collagenase was used with a single room temperature incubation of 8 minutes followed by mixing for 1 minute per well prior to resuspension.

Published collagenase protocols typically suggest single, short incubations with varying concentrations of collagenase with the proviso that the conditions may have to be optimized for the cell line of interest.

Results

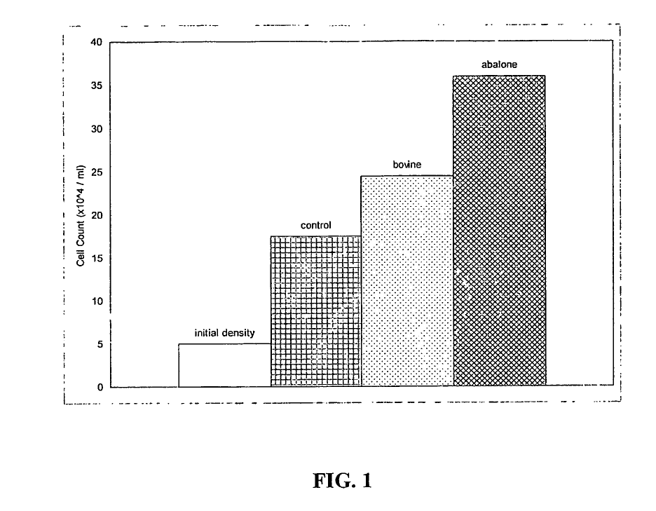

The growth of the cultures as measured by daily cell counts are shown graphically in FIG. 1. The control cells (grown on uncoated plastic) showed a slower but more linear growth, reaching a higher maximum cell number. The growth on collagen coated surfaces was initially more rapid as was the onset of differentiation.

Cells on the coated surfaces quickly display a flattened appearance associated with attachment and exhibit a greater degree of organization, with cells aligned in band-like structures (FIG. 2). Cells on the uncoated surfaces took longer to flatten out and then appeared to be randomly arranged.

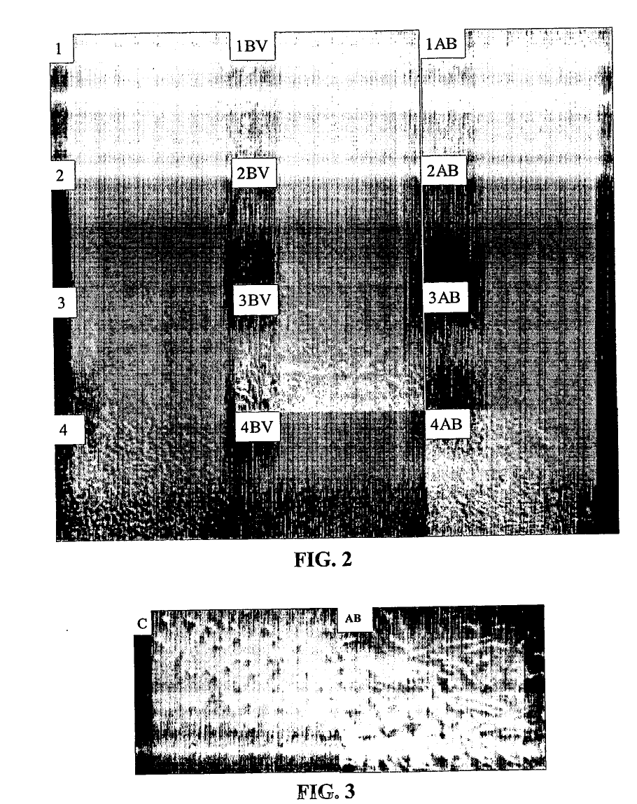

Following long term culture the cells on the collagen coated surface are of higher number and still of flattened appearance. Cells on the uncoated surface are fewer and have become rounded (FIG. 3). Cell viability and differentiation are maintained longer on collagen coated surfaces.

The cells grown on abalone collagen have shown a measurably higher growth rate than those on bovine collagen and appear to exhibit greater organization.

### REFERENCES

The references listed below have their disclosure incorporated herein through reference:

Francois C. J. and Glincher M. J. (1967) Biochim. Biophys. Acta 133, 91.

Helseth D. L Jr and Veis A (1981) J. Biol. Chem. 256, 7118-7128.

Hofmann H, Fietzek, P. P and Kuhn K (1980) J. Mol Biol. 141, 293-314.

Katzman R. L and Kang A. H (1972) J. Biol. Chem 247, 5486.

Kefalides N. A (1971) Biochem Biophys Res. Commun. 46, 226.

U.K. Laemmli (1970) Nature 227, 680-685.

Laurain G, Delvincourt T, and Szymanowicz A. G. (1980) FEBS Letter, 120, 44-48.

Lewis M. S and Piez K. A. J. (1964) Biol. Chem. 239, 336.

McClain P. E., Creed G. J., Wiley E. R. and Gerrits R. J. (1970) Biochim.Biophys Acta 221, 349.

Miller E. J Biochemistry (1971) 10, 1652.

Miller E. J., Martin G. R., Piez K. A and Powers M. J. J. Biol. Chem (1967) 242, 5481.

Piez K. A Biochemistry (1965) 4, 2590.

Piez, K. A, Eiger A, and Lewis M. S (1963) Biochemistry 2, 58.

Piez K. A (1984) Molecular and aggregate structures of the collagens. In Extracellular Matrix Biochemistry (Piez, K. A and Reddi A. H. eds) pp 1-39, Elsevier New York.

Prockop D. J and Kivirikko K. I (1995) Annu. Rev. Biochem 64, 403-434.

Trelstad R. I. Kang A. H Igarashi S. and Gross J. (1970) Biochemistry 9, 4993.

1. A process for isolating a soluble, native collagen from a marine invertebrate, comprising the steps of:
1) treating a collagen-containing portion of the marine invertebrate with a weak acid solution in order to solubilise native collagen fibrils;
2) centrifuging the resultant slurry to remove tissue particulates;
3) adjusting the pH of the supernatant in order to precipitate collagen by addition of a base;
4) collecting the precipitated collagen;
5) resuspending the precipitated collagen; and
6) performing buffer exchange against water using an ultrafiltration membrane.

2. A process as claimed in claim 1 wherein the pH adjustment takes place after the collagen-containing portion has been in contact with the weak acid solution for 1 to 20 days.

3. A process as claimed in claim 2 wherein the pH adjustment takes place after 3 to 6 days.

4. A process as claimed in claim 3 wherein the pH adjustment takes place after 6 days.

5. A process as claimed in claim 1 wherein the pH adjustment is made by the gradual addition of a strong base.

6. A process as claimed in claim 5 wherein the pH adjustment is made by the gradual addition of 1M sodium hydroxide.

7. A process as claimed in claim 5 wherein the pH adjustment is made over a period of 1 to 10 hours in a coldroom.

8. A process as claimed in claim 7 wherein the pH adjustment is made over a period of 2 to 6 hours.

9. A process as claimed in claim 8 wherein the pH adjustment is made over a period of 3 hours.

10. A process as claimed in claim 7 wherein the pH adjustment is made with continuous stirring.

11. A process as claimed in claim 1 wherein the marine invertebrate is abalone.

12. A process as claimed in claim 11 wherein the marine invertebrate is selected from the group consisting of the black lip abalone, Haliotis ruber, the brown-lip abalone Haliotis conicopora and the green-lip abalone, Haliotis laevigata, or Roe's abalone, Haliotis roei.

13. Collagen when prepared by the process of claim 1.

14. A process for preparing gelatin comprising heating the collagen of claim 13.

15. The use of the collagen of claim 13 in place of collagen isolated from a land vertebrate or gelatin prepared from the collagen of a land vertebrate.

16. The use as claimed in claim 15 as a cosmetic ingredient, in the form of injectable collagen, in biomedical devices, as a pharmaceutical substance, as a surface

17. A cell culture vessel coated with collagen according to claim 13.

18. A cosmetic composition comprising collagen according to claim 13.

19. A biomedical device comprising collagen according to claim 13.

20. A pharmaceutical composition comprising collagen according to claim 13.

21. A food comprising collagen according to claim 13.

22. A beverage prepared using collagen according to claim 13 as a fining agent.

23. Gelatin when prepared by the process of claim 14.

24. A capsule for pharmaceutical comprising gelatin according to claim 23.

25. A method of preparing a dried native collagen film suitable for use as a cell culture medium, comprising the steps of:
(1) providing a solution of native collagen derived from abalone;
(2) applying the solution to a surface so as to coat the surface with a film of native collagen derived from abalone; and
(3) drying the film to form a dried collagen film on the surface.

26. A method as claimed in claim 25 wherein the surface is a surface of a cell culture vessel.

27. A method as claimed in claim 26 wherein the surface is a well within a multi-well cell culture plate.

28. A cell culture vessel coated at least in part with native collagen derived from abalone.

29. A cell culture vessel as claimed in claim 28 wherein the vessel is a multi-well cell culture plate wherein at least one well is coated with dried native collagen derived from abalone.

30. A cell culture medium comprising native collagen derived from abalone.

31. A cell culture medium as claimed in claim 30 in the form of a dried film.

32. A cell culture medium as claimed in claim 30 in the form of a gel.

33. A kit for cell culture comprising:
(a) native collagen derived from abalone; and
(b) a weak acid solution for admixture with the native collagen derived from abalone to enable preparation of a solution of native abalone collagen.

34. A method of culturing cells comprising the steps of:
(1) providing a cell culture medium as claimed in claim 30;
(2) applying cells to be cultured to the culture medium; and
(3) culturing the cells.

35. A method as claimed in claim 34, further comprising the step of:
(4) achieving a higher growth rate for the cells applied to and cultured on the abalone collagen culture medium than for the same kind of cells applied to and cultured on a bovine collagen culture medium.

36. A method of culturing cells consisting essentially of:
(1) providing a cell culture medium as claimed in claim 30;
(2) applying cells to be cultured to the culture medium; and
(3) culturing the cells.

37. A method as claimed in claim 36, further including the step of:
(4) achieving a higher growth rate for the cells applied to and cultured on the abalone collagen culture medium than for the same kind of cells applied to and cultured on a bovine collagen culture medium.
